(19) United States
(12) Patent Application Publication
(10) Pub. No.: US 2026/0144684 A1
(43) Pub. Date: May 28, 2026

(54) OCCLUSIVE CHEST DRESSING

(71) Applicant: Timothy A. Caldwell, Sanford, FL (US)

(72) Inventor: Timothy A. Caldwell, Sanford, FL (US)

(21) Appl. No.: 19/374,247

(22) Filed: Oct. 30, 2025

Related U.S. Application Data
(60) Provisional application No. 63/724,193, filed on Nov. 22, 2024.

(51) Int. Cl. A61F 13/14; A61M 1/04

(52) U.S. Cl. CPC A61F 13/143; A61M 1/04

(57) ABSTRACT

A chest dressing includes an air and fluid impermeable main body having a diverter that is positioned centrally along the bottom surface and a plurality of fluid drains extending outward therefrom. Each of the fluid drains include an opening along a distal end that removes blood and air from the bottom surface of the main body through to the top surface of the main body. The bottom surface including a layer of skin safe adhesive that is positioned between the plurality of fluid drains and along the entire outer periphery of the main body. A removable protective cover is positioned against the adhesive on the bottom surface of the main body, and the main body and protective cover include living hinges to permit the device to fold twice in order to reduce the size of the device by 75% when being stored.

### CROSS-REFERENCE TO RELATED APPLICATIONS

This application claims the benefit of U.S. Application Ser. No. 63/724,193 filed on Nov. 22, 2024, the contents of which are incorporated herein by reference.

### TECHNICAL FIELD

The following disclosure contemplates various embodiments of an occlusive chest dressing for creating an airtight seal over a thoracic injury to prevent air from entering the pleural cavity during respiration.

### BACKGROUND

The statements in this section merely provide background information related to the present disclosure and may not constitute prior art.

Traumatic injuries to the thoracic cavity often result in sucking chest wounds which are fatal unless they are treated rapidly. This is because sucking chest wounds breach the thoracic cavity and allow pressure equalization with the outside atmosphere, thus eliminating the natural internal vacuum required for the lungs to function normally. First response treatment for these wounds involves covering the wound with an impermeable material, such as a piece of plastic, and providing a one-way valve to allow air to escape from the wound, while preventing air or other materials from entering the thoracic cavity through the wound.

Although sucking chest wounds can occur anywhere, they are most common in battlefield situations where soldiers sustain gunshot wounds to the chest. As such, there are several known types of commercially available chest wound patches.

For example, one of the most popular type of chest wound patches is described in U.S. Pat. No. 7,504,549 to Castellani provides for a patch having a centrally located chamber that is connected to an open ended drainage channel that extends through the distal end of one side of the patch. Although useful, these patches suffer from several practical drawbacks.

First, because the drainage channel runs from the central chamber to the side of the patch and exits beneath the top surface of the patch, it is not possible to provide adhesives along the entire outer periphery of the patch to provide a 100% seal. As such, it can be difficult to secure these patches onto chest wounds where blood or other liquids are present.

Moreover, in these areas where the open ended channel exits the bottom of the patch, blood and other such materials traveling along the channel are able to make contact with the glue that borders the open end of the channel on the bottom surface of the patch. As this happens, the glue becomes diluted and the size of the channel expands until the patch loses its seal with the chest cavity, thus reducing or eliminating the effectiveness of the patch.

Second, owing to the orientation of the central chamber and the drainage channel, extreme care must be taken when attempting to fold or bend these devices, as this can cause the glue to make contact with the channel thus rendering the device unusable. As such, it is recommended that medics do not fold or otherwise bend these products, and if it is necessary to do so, that they only bend them a single time (in half). As a result, these conventional devices occupy a disproportionally large amount of space within a medical backpack, as a standard 8″×8″ patch can only be folded to a size of 8″×4″.

Finally, when used in battlefield situations, it can be extremely difficult for combat medics to quickly identify the orientation of the patch and to effectively engage and remove the adhesive cover to apply the patch onto the patient. This is especially true if the rescue is occurring at night where night vision instruments are being utilized.

The present invention, directed to an occlusive chest dressing differs from the conventional art in a number of aspects. The manner by which will become more apparent in the description which follows, particularly when read in conjunction with the accompanying drawings.

### SUMMARY OF THE INVENTION

The present invention is directed to a chest dressing. One embodiment of the present invention can include a main body having a top surface, a bottom surface, and a plurality of sides. A diverter can be positioned centrally along the bottom surface and can be connected to a plurality of fluid drains that extend outward toward each of the plurality of sides. Each of the plurality of fluid drains can include an opening that extends from the bottom surface to the top surface of the main body.

In one embodiment, the bottom surface can include a layer of skin safe adhesive and each of the diverter and plurality of fluid drains can be formed from the absence of adhesive material. In one embodiment, the dressing can include a removable protective cover that is positioned along the bottom surface of the main body and is in contact with the adhesive.

In one embodiment, each of the main body and the protective cover can include a plurality of living hinges that are aligned complementary to each other to permit the main body and protective cover to fold twice along a first and second axis to reduce the overall size of the device by 75% when being stored.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a top view of a chest dressing that is useful for understanding the inventive concepts disclosed herein.

FIG. 2 is a bottom view of the main body of the chest dressing in accordance with one embodiment of the invention.

FIG. 3 is a bottom view of the main body and protective cover of the chest dressing in accordance with one embodiment of the invention.

FIG. 4 is a top view of the chest dressing in accordance with one embodiment of the invention.

FIG. 5 is a bottom view of the chest dressing in accordance with one embodiment of the invention.

FIG. 6 is a top view of the chest dressing in a partially folded orientation, in accordance with one embodiment of the invention.

FIG. 7 is a top view of the chest dressing in a fully folded orientation, in accordance with one embodiment of the invention.

FIG. 8 is a top view of the chest dressing in operation, in accordance with one embodiment of the invention.

FIG. 9 is a cross-sectional view of the chest dressing of FIG. 8, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

Any element in a claim that does not explicitly state “means” for performing a specified function or “step” for performing a specified function should not be interpreted as a “means” or “step” clause as specified in 35 U.S.C. 112.

### Definitions

As described herein, a “unit” means a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances, or the like.

As described throughout this document, the term “removably secured” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

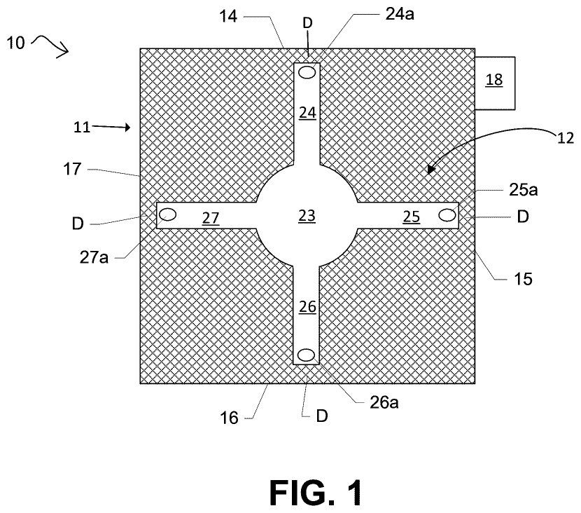

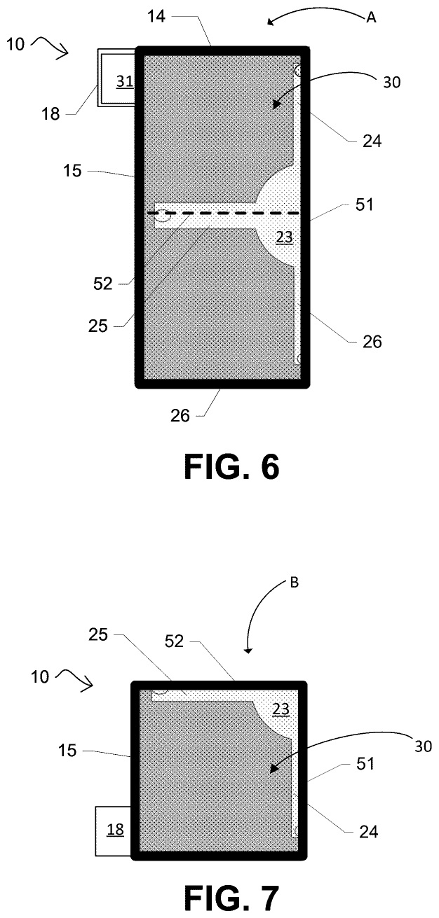

FIGS. 1-9 illustrate one embodiment of an occlusive chest dressing 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

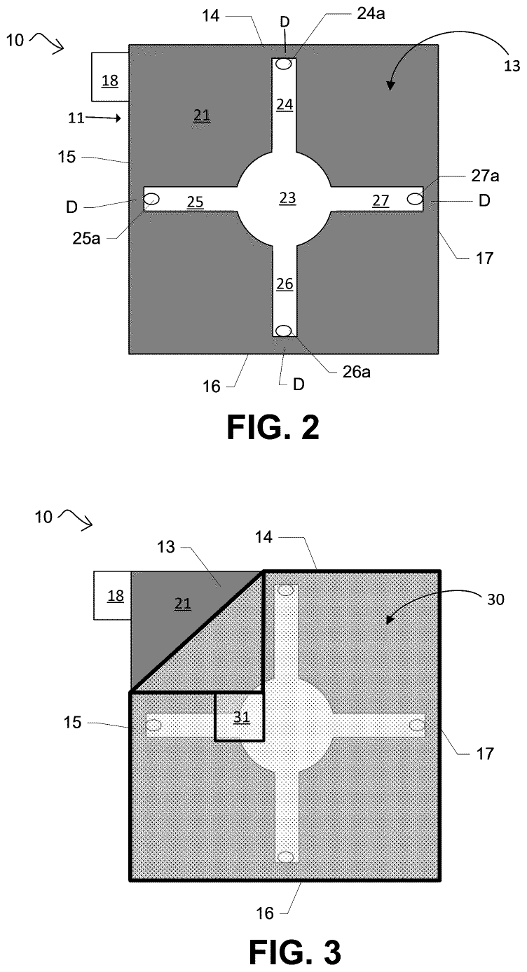

FIGS. 1 and 2 illustrate one embodiment of the occlusive chest dressing device 10 that includes a generally square shaped main body 11 having a top surface 12, a bottom surface 13, and sides 14, 15, 16 and 17. In the preferred embodiment, the main body 11 can be formed from a thin (e.g., 0.2 mm to 0.5 mm) piece of malleable plastic that is air and fluid impermeable. Of course, other embodiments are contemplated wherein the main body includes a different shape, and/or is constructed from any number of other types of airtight and fluid impermeable materials.

In one embodiment, a tab 18 can be positioned along one edge of the main body. The tab can provide a user with a place to securely grip the main body without touching the adhesive on the bottom surface. In one embodiment, the tab 18 can be constructed from, lined with, or otherwise include an infrared reflective material that reflects infrared wavelengths when illuminated by light sources such as night vision goggles or other IR systems. Such a feature being advantageous for use in battlefield situations to aid combat medics wearing such equipment to be able to securely locate, grip and apply the device onto a patient.

In one embodiment, adhesive 21 can be disposed along the bottom surface 13 of the main body 11. As described herein, the adhesive 21 can include any type of skin adherent material such as a hydrogel polymeric composition, for example, that is safe for use on human skin, is capable of molding to the shape of the body part to which it is secured, and that can be applied and reapplied multiple times without losing its adhesive properties. Of course, any number of other non-antigenic and non-allergenic adhesive materials capable of flexing with the movement of the patient and securing the device onto a patient to provide an airtight seal over a wound may also be utilized.

In one embodiment, a generally circular-shaped diverter 23 can be positioned centrally along the bottom surface 13 of the main body 11, and a plurality of fluid drains 24-27 can extend outward from the diverter 23 along the bottom surface 13. In the preferred embodiment, each of the diverter 23 and drains 24-27 can be formed along the main body by the absence of adhesive 21 on the bottom surface of the main body; however other embodiments are contemplated wherein each of the components 23-27 are formed by protruding sections of main body material to form physical barriers against which the adhesive abuts.

In either instance, the distal ends of each of the drains 24-27 can terminate a set distance D from the outer edge of the sides 14-17 of the main body to allow the adhesive material 21 to be located along the entire outer edge of each side of the main body. Such a feature is important as it ensures a complete and uniform seal between the entire outer periphery of the main body and the patient to which the device is secured.

In the preferred embodiment, the set distance D will be between 0.25 and 0.5 inches. This amount being suitable for securely adhering the main body onto human skin to create the desired impermeable seal. Of course, other embodiments are contemplated wherein the set distance is different from this amount and/or wherein the central diverter includes a different shape.

In one embodiment, a plurality of drain openings 24a-27a can be provided along the main body at locations adjacent to the distal ends of the drains 24-27, respectively. Each of the openings can be formed through the malleable main body material to permit air and fluids located within the drains on the bottom surface 13 of the main body to pass through to the top surface 12 of the main body as they exit the chest cavity of the patient.

Owing to the malleable nature of the main body material, the shape and/or orientation of each opening on each drain can change to transition between an open position and a closed position. For example, in the closed position each opening will remain flat against the skin of the patient, thus forming an impermeable seal that prevents air or fluids from entering the drain through the opening. Conversely, as air and fluids located within a specific drain reaches its respective opening, the main body material at that location will rise off the skin due to the lack of adhesive, thus permitting the air or fluid to be expelled from the drain via the opening (i.e., the open position).

As shown best at FIG. 3, the device 10 can include a removable protective cover 30 with integrated tab 31 having a shape and size that is complementary to the shape and size of the main body 11 and tab 18 described above. The cover can be constructed from a piece of nonstick plastic such as PTFE, for example, and can be positioned along the bottom surface 13 to protect and preserve the adhesive 21 until the device is ready to be used.

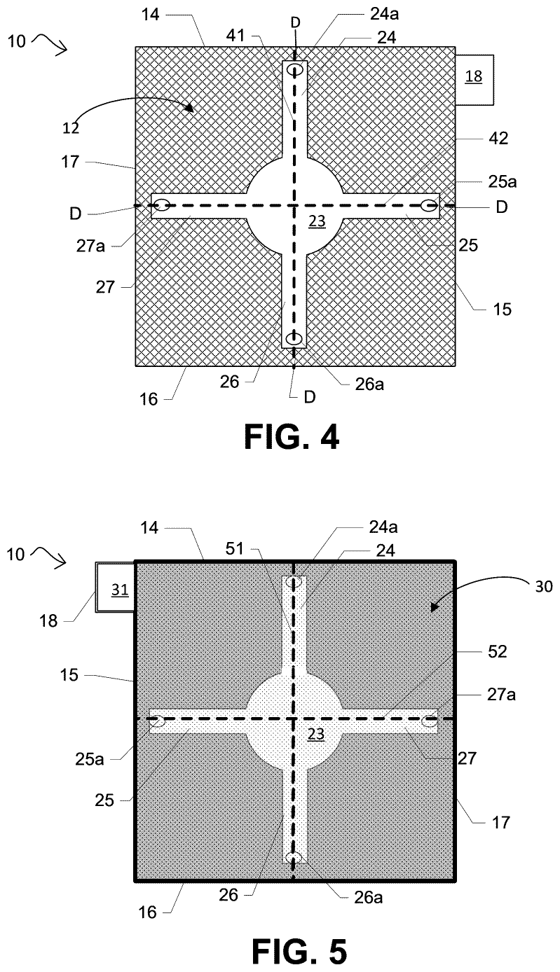

FIGS. 4-7 illustrate one embodiment of the device 10 that includes a plurality of living hinges 41-42 and 51-52 that are provided along the main body 11 and protective cover 30, respectively. As shown, living hinges 41 and 51 can extend completely between walls 14 and 16 of the main body (e.g., a first axis) at a location centered within drains 24 and 26. Likewise, living hinges 42 and 52 can extend completely between walls 15 and 17 of the main body (e.g., a second axis) at a location centered within drains 25 and 27.

In one embodiment, the living hinges can include or can be created by one or more indentations in the main body and cover materials. In this regard, it is noted that the indentations do not extend completely through the main body, and thus ensures the main body remains airtight.

The location and orientation of the living hinges along the drains is an important feature for at least two reasons. First, because the diverter 23 and drains 24-27 do not have adhesives, the main body is able to bend and fold along the drains without affecting the adhesive layer other than the amount within the distal spaces D along the edges of the main body. Such a feature both preserves the adhesive and ensures that no adhesive material will make contact along the drains or diverter which would reduce the effectiveness of the drain during use.

Secondly, the perpendicular arrangement of the living hinges allows the device to be evenly folded two times in both width-e.g., between ends 15 and 17 as shown by arrow a, and in height e.g., between ends 14 and 16 as shown by arrow b. Such a feature allows the resulting device to be packaged and shipped with a footprint that is 1/4 the size of the device when unfolded and ready to be used. To this end, when the unfolded size of the device is 5″×5″, the folded and packaged size of the device is only 2.5″×2.5″. Such a feature is extremely important, as it ensures the device does not occupy a large portion of the limited space allocated within a medic's emergency kit.

In operation, the main body 11 is designed to be positioned onto the skin of a patient so as to cover an open or sucking chest wound. As such, the device can be used by a medic by first removing the packaging, unfolding the device, locating the tab 18, and removing the protective cover 30.

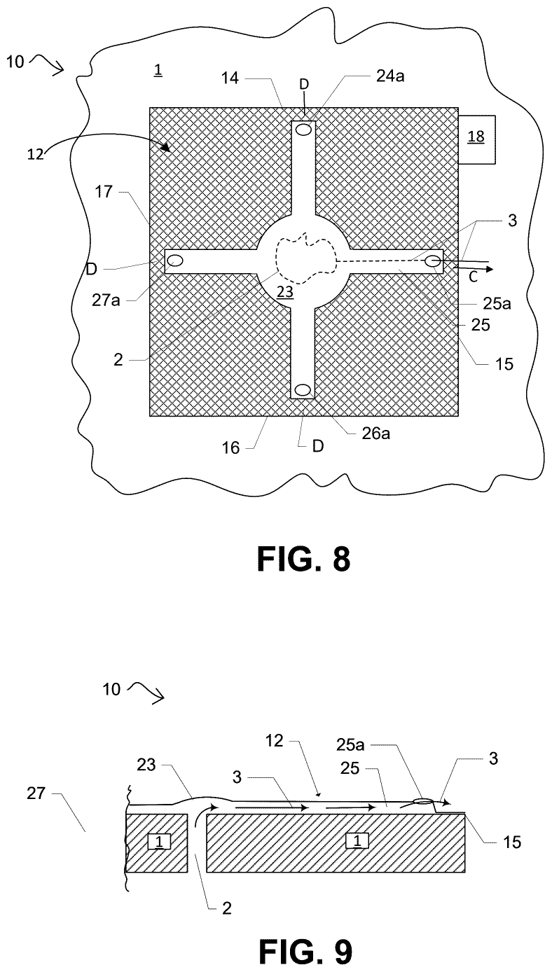

As shown at FIGS. 8 and 9, device 10 can be positioned onto the skin 1 of the patient with the drain diverter 23 located directly over the patient's chest wound 2.

When so positioned, the entire outer periphery of the main body (e.g., the entirety of sides 14-17) will be secured against the patient's chest via the adhesive to create an airtight seal. Next, as the patient breathes, blood 3 exiting the wound will make contact with the diverter 23 and will be routed along one or more of the drains 24-27 on the bottom surface of the main body. As the blood reaches the opening(s) 24a-27a, it will pass through the respective opening to transition from the bottom surface of the main body to the top surface of the main body 11 where it will be expelled.

Although described above as including a specific shape, size, material, and/or number of drains, this is for illustrative purposes only. As such, the inventive concepts can include any number of different shapes, sizes, materials and/or drains to achieve the functionality described herein.

As to a further description of the manner and use of the present invention, the same should be apparent from the above description. Accordingly, no further discussion relating to the manner of usage and operation will be provided.

As described herein, one or more elements of the above noted device and system can be secured together utilizing any number of known attachment means such as, for example, screws, glue, compression fittings, and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

Any element in a claim that does not explicitly state “means” for performing a specified function or “step” for performing a specified function should not be interpreted as a “means” or “step” clause as specified in 35 U.S.C. 112. The corresponding structures, materials, acts, and equivalents of all means or step plus function elements in the claims below are intended to include any structure, material, or act for performing the function in combination with other claimed elements as specifically claimed.

The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A chest dressing device, comprising:
a main body having a top surface, a bottom surface, and a plurality of sides;
a layer of adhesive that is positioned along the bottom surface;
a diverter that is positioned centrally along the bottom surface; and
a plurality of fluid drains that are positioned along the bottom surface,
wherein the layer of adhesive is configured to secure the main body onto the skin of a patient, wherein the diverter is configured to be positioned adjacent to a chest wound of the patient, and each of the plurality of fluid drains are configured to remove blood from the chest wound.

2. The device of claim 1, wherein the main body is constructed from a liquid impermeable and air impermeable material.

3. The device of claim 1, wherein the plurality of sides includes a first side, a second side, a third side and a fourth side.

4. The device of claim 3, wherein the plurality of fluid drains includes a first drain, a second drain, a third drain and a fourth drain.

5. The device of claim 4, wherein the first drain extends from the diverter toward the first side, the second drain extends from the diverter toward the second side, the third drain extends from the diverter toward the third side, and the fourth drain extends from the diverter toward the fourth side.

6. The device of claim 5, wherein the main body is configured to fold in half along a first axis.

7. The device of claim 6, wherein the main body in the half folded position is configured to fold in half again along a second axis.

8. The device of claim 7, wherein the first axis and the second axis are oriented perpendicular to each other.

9. The device of claim 5, further comprising:
a first living hinge that extends between the first side to the third side.

10. The device of claim 9, further comprising:
a second living hinge that extends between the second side to the fourth side.

11. The device of claim 1, further comprising:
a main body pull tab that is positioned along one of the plurality of sides.

12. The device of claim 11, further comprising:
a protective cover that is configured to be positioned against the adhesive material along the bottom surface of the main body.

13. The device of claim 12, wherein the protective cover includes a pull tab that is positioned at a location complementary to a location of the main body pull tab.

14. The device of claim 1, wherein the main body is configured to allow air to escape from the chest through the diverter and plurality of fluid drains, and to prevent air from entering the chest wound through the diverter and plurality of fluid drains.
